(19) United States
(12) Patent Application Publication
(10) Pub. No.: US 2019/0125686 A1
(43) Pub. Date: May 2, 2019

(54) BONE REGENERATION PROMOTING COMPOSITION AND BONE REGENERATION PROMOTING APPARATUS

(71) Applicant: AQUA ZEST CORPORATION, Kawasaki-shi, Kanagawa (JP)

(72) Inventors: Ichiro KAMEI, Tokyo (JP); Kenichi ICHIHASHI, Hyogo (JP)

(21) Appl. No.: 16/082,463

(22) PCT Filed: Mar. 6, 2017
(86) PCT No.: PCT/JP2017/008860

§ 371 Date: Dec. 31, 2018

(30) Foreign Application Priority Data
Mar. 6, 2016 (JP) 2016-042912

(51) Int. Cl. A61K 9/50; A61P 19/08; A61K 33/00; A61K 9/00; A61K 9/46

(52) U.S. Cl. CPC A61K 9/50; A61P 19/08; A61K 9/0007; A61K 9/0053; A61K 33/00

(57) ABSTRACT

[Problem] To provide a composition and apparatus for promoting the regeneration of bone, specifically cartilage, as well as a bone regeneration promoting method.

[Solution] A bone regeneration promoting composition that includes, as active ingredients, nanobubbles of hydrogen and/or of oxygen and nitrogen. A method for promoting bone regeneration by combining the bone regeneration composition, hormesis, and spatial pressure-based therapy. A sealable chamber apparatus, the apparatus being provided with a supply means for a gas mixture of hydrogen, oxygen, and nitrogen, and a control means for the internal chamber atmospheric pressure. Additionally provided is a method for promoting bone regeneration using the apparatus.

### TECHNICAL FIELD

The present invention relates to a composition for promoting bone regeneration and a bone regeneration promoting device, and more particularly to a composition for promoting cartilage regeneration and a cartilage regeneration promoting device.

### BACKGROUND ART

Since cartilage tissue does not have a tissue nutrition system by blood circulation, it has poor tissue repair ability, and once damaged it is difficult to repair or regenerate it. If articular cartilage repair or regeneration is not sufficient, osteoporosis or osteoarthritis may develop.

Conventionally, as a method for preventing or treating the above-mentioned symptoms, injection of high molecular weight hyaluronic acid into a joint locality was common. Recently, as a component effective for prevention or treatment, collagen peptide, joint strengthened beverage containing glucosamine salt (Patent Document 1), an agent for ameliorating rheumatoid arthritis or osteoarthritis comprising an effective component of a tripeptide having an amino acid sequence of Gly-X-Y, which is obtained by degrading a collagen component or a gelatin component with a collagenase enzyme (Patent Document 2), and an agent for treating oral joint disorder or a functional food characterized by containing at least one selected from collagen and collagen peptide, at least one selected from aminosugar, mucopolysaccharide and uronic acid (patent literature 3) are also known. However, depending on the oral administration of glucosamine for 6691 women overseas from 50 to 60 years of age, there is no significant effect on ingestion and onset, and the effect of prevention of onset has not been proven.

In recent years, it has become possible to prepare chondrocytes from iPS cells and the like, but it is still expected that regenerative medicine will take a considerable amount of time to put it into practical use, and treatment costs will also be high.

Bone regeneration by low irradiation therapy (hormesis) and atmospheric pressure changing therapy is also attempted, but sufficient cartilage regeneration effect has not been obtained.

On the other hand, it has been reported that the nanobubble-containing composition has various effects, but further development of a new application has been demanded (Patent Document 4).

Idiopathic femoral head necrosis is one of hip joint diseases in which the bone tissue of the femoral head is necrotic due to a decrease in blood flow and the joint is deformed and destroyed. The theories of causes include blood fat, blood vessel abnormality, and blood clotting abnormality, the mechanism of onset has not been elucidated accurately. Therefore, there is no precautionary measure, mostly shifts to replacement of artificial femoral heads or artificial joints, and it is designated as refractory disease. For this reason, agents for prevention and treatment, and methods for prevention and treatment for sudden femoral head necrosis have been demanded.

Therefore, development of a cartilage regenerating device and a composition for regenerating cartilage has been demanded.

### RELATED ART DOCUMENTS

### Patent Documents

Patent literature 1: Japanese Laid-Open Patent Publication No. 2002-125638
Patent literature 2: Japanese Laid-Open Patent Publication No. 2002-255847
Patent literature 3: Japanese Laid-Open Patent Publication No. 2003-48850
Patent literature 4: International Publication No. 2015-099201

### SUMMARY OF THE INVENTION

A bone regeneration promoting composition that includes nanobubbles of hydrogen and/or of oxygen and nitrogen as active ingredients. A method for promoting bone regeneration by combining the bone regeneration composition, hormesis, and spatial pressure-based therapy. A sealable chamber apparatus, the apparatus being provided with a supply means for a gas mixture of hydrogen, oxygen, and nitrogen, and a control means for the internal chamber atmospheric pressure. Additionally provided is a method for promoting bone regeneration using the apparatus.

### Problem to be Solved by the Invention

Compositions and apparatus for promoting regeneration of bone, particularly cartilage are provided.

### Means for Solving the Problem

According to the present invention, a composition for promoting bone regeneration comprising nanobubbles of hydrogen, and/or oxygen and nitrogen as an active ingredient is provided. In this case, the composition for promoting bone regeneration includes not only a medical composition but also a food composition.

In one embodiment of the present invention, one or more methods selected from the group of hormesis, space pressure changing therapy and magnetic therapy can be combined with the composition for promoting bone regeneration. That is, a bone regeneration promoting composition to promote bone regeneration by combination of these is provided.

A cartilage regeneration method is also provided comprising a step of administering the bone regeneration composition of the present invention to a subject, a step of irradiating radiation, and a step of pressurizing. More specifically, a cartilage regeneration method having a step of orally administering 500 mL to 1000 mL of the bone regeneration composition of the present invention, a step of irradiating low dose of radiation with hormesis, and a step of pressurizing by spatial pressure changing therapy is provided.

According to the present invention, a composition for promoting bone regeneration for combination therapy of hormesis and space pressure changing therapy is provided, which contains hydrogen nano bubbles as an active ingredient.

One embodiment of the present invention provides a composition for promoting bone regeneration for combination therapy of hormesis and spatial pressure change therapy, which comprises nanobubbles of hydrogen, and/or oxygen and nitrogen as an active ingredient. Also, there is provided a method for promoting bone regeneration comprising the steps of administering a bone regeneration promoting composition containing nanobubbles of hydrogen and/or oxygen and nitrogen as an active ingredient, a step of irradiating a low dose of radiation and a step of pressurizing by space pressure changing therapy. As a method for administering the bone regeneration-promoting composition of the present invention to the human body, oral administration is preferable, but not limited thereto, administration by injection (including drip) or the like may be used. In short, any method may be used as long as bone regeneration is promoted.

In one embodiment of the present invention, the composition for promoting bone regeneration is a composition for promoting cartilage regeneration.

According to another aspect of the present invention, there is provided a cartilage regeneration system comprising the bone regeneration-promoting composition, a device for hormesis and a device for space pressure changing therapy. In this case, the instrument for hormesis may be a small appliance capable of local irradiation or a sheet with a radioactive substance arranged so as to irradiate the whole body.

According to another aspect of the present invention, an operation method of an atmospheric pressure changing machine to pressure to 1.1 to 1.5 atm at the ratio of hydrogen:oxygen:nitrogen is 0.1 to 4.0:20 to 50:79.9 to 46.

According to another aspect of the present invention there is provided a sealable capsule device comprising means for supplying a mixture of hydrogen, oxygen and nitrogen, and device for controlling the pressure in the capsule. Also provided is a method of promoting bone regeneration using such a device.

### Effects of the Invention

According to the present invention, it is possible to regenerate part or all of bone, particularly cartilage.

### BRIEF DESCRIPTION OF THE DRAWINGS

FIG. 1 is a photograph showing a tissue repair from a bone head necrosis of a 38 year old male.

FIG. 2 is a photograph showing improvement of right femoral head necrosis of a 72-year-old man.

FIG. 3 is a photograph showing improvement of right femoral head necrosis of a 76-year old female.

FIG. 4 is a photograph showing an improvement in left femoral neck fracture of an 80 year old female.

FIG. 5 is a photograph showing improvement of left femoral head necrosis of a 27-year-old male.

FIG. 6 is a photograph showing improvement of 82-year old right knee osteoarthritis.

FIG. 7 is a photograph showing improvement of a 71-year-old female left knee osteoarthritis.

FIG. 8 is a photograph showing improvement of osteoarthritis inner articular cartilage defect of a 75-year-old female.

FIG. 9 is a photograph showing improvement of osteoarthritic medial articular cartilage defect of an 80-year old female.

FIG. 10 is a photograph showing improvement of a right knee femoral cartilage defect of a 49-year-old male.

FIG. 11 is a diagram of an apparatus for performing atmospheric pressure changing therapy.

FIG. 12 is a diagram of another embodiment of a device for implementing atmospheric pressure changing therapy.

FIG. 13 is a photograph showing improvement of left knee osteoarthritis of a 84 year old female.

FIG. 14 is a photograph showing improvement of left knee osteoarthritis of a 68-year-old male.

FIG. 15 is a photograph showing improvement of patellofemoral arthritis of a 75-year-old female.

FIG. 16 is a photograph showing improvement of left knee osteoarthritis of a 73-year-old female.

FIG. 17 is a photograph showing improvement of 83-year-old left knee osteoarthritis.

FIG. 18 is a photograph showing improvement of knee left femor internal condyle soft bone defect in a 75-year-old female.

FIG. 19 is a photograph showing improvement of right femoral head necrosis of an 87 year old female.

### DESCRIPTION OF THE EMBODIMENTS

The present invention is characterized in that cartilage regeneration is promoted by ingesting water containing hydrogen when low-dose local radiation irradiation therapy (radiation hormesis) and atmospheric pressure changing therapy are used in combination. That is, the present invention provides a composition for promoting cartilage regeneration ingested during hormesis and atmospheric pressure changing therapy. However, for example, in the case of a person who is highly sensitive to radiation or a person whose ear has disorder and who can not tolerate high pressure, a combination of any of atmospheric pressure changing therapy or hormesis, and the bone regeneration promoting composition of the present invention may be used in combination.

Radiation hormesis is to stimulate bioactivity by a small amount (low radiation dose) of ionizing radiation which is harmful in a large amount (high dose), and to cause adaptive response that provides resistance to subsequent high dose irradiation. In the present specification, hormesis may be used in the sense of low-dose radiation therapy. It has been reported that by radiation hormesis, SOD and glutathione are increased, active oxygen scavenging ability is increased, or apoptosis of DNA damaged cells is progressed by activation of P53 cancer suppressing gene (Takeo Onishi, J. Radiat Res. 151:368, 1999). In addition, immune system cells are regarded to be activated, and immune function activation of killer T cells in high radon room has been reported (Takanori Yamaoka, J. Radiat Res. 46:21, 2005).

The inventors have found that cartilage regeneration becomes possible by combining hormesis, atmospheric pressure changing therapy and the cartilage regeneration promoting composition of the present invention, and have completed the invention.

The hormesis to be carried out when using the cartilage promoting composition of the present invention irradiates low dose radiation to affected part for 50 to 70 minutes so that it is 10 to 274 microsievert, more preferably 10 to 100 microsievert, particularly preferably 20 to 28 microsieverts.

In the present invention, the radiation source material used as a hormesis material comprises a radioactive substance (specifically, a radiation source adjusted substance) and a binder. As a radioactive substance, a rare earth mineral containing natural radioactive element uranium-thorium is used, and among the ores used as a raw material in the rare earth industry, a relatively large amount of thorium (Th) and uranium (U) is contained thing, for example, monazite (chemical formula (Th, RE) PO₄, ThO₂ 6.0%, U₃O₈ 0.3%, (RE)₂O₃ 60%, P₂O₅ 28%), or Xenotime (chemical formula YPO₄, U₃O₈ 1.0%, ThO₂ 1.0%, (RE)₂O₃ 50%, P₂O₅ 45%) are common. However, the type of rare earth ore is not particularly specified. Generally, what is obtained by diluting a natural radioactive substance with other substance (hereinafter referred to as a radiation source adjusted substance) is used. For example, a natural radioactive rare earth mineral diluted with white ceramic materials (quartz, feldspar, clay, mullite, zircon, zirconia, alumina, zeolite, etc.) may be used. Monazite and zircon may be used, and those mixed at a ratio of monazite:zircon=30:70 may be crushed to be used. The use amount of the radiation source adjusted substance can be determined experimentally. However, those containing these radionuclides, which are nuclear source materials, have restrictions on their use. If their concentrations are 370 Becquerel/g (in current regulations, Thorium containing %+3×uranium containing % 1.8%) or more, a notification is required. Therefore, in terms of being usable even without such notification, the mixing ratio of the radiation source material is preferably less than 2.0% by weight, more preferably less than 1.8% by weight in terms of the content of thorium oxide. That is, although not restricted, the compounding ratio thereof is preferably from 0.3 to 2.0% by weight, more preferably from 0.3 to 1.8% by weight in terms of the content of thorium oxide. As commercially available products, Mino pigment made Iomix can be used. As the hormesis sheet used in the present invention, a sheet containing radium 226 can be suitably used, but it is not limited to this, and it can be used as long as it can irradiate a necessary amount of radiation and has a therapeutic effect.

It is preferable that the radiation source material is fine powder of finely divided particles, and it is generally preferable that the average particle diameter is 10 micrometer or less. More preferred is an average particle diameter of about 0.5 to 1 micrometer. And the finer the particle size, the more effectively it can absorb energy rays due to radioactive decay of natural radioactive elements. Generally, the radiation source material composed of radioactive minerals can be blended in a proportion of 5 to 60% by weight, preferably 5 to 30% by weight with respect to the whole.

The form of the therapeutic instrument including the radiation source material is not particularly limited and may be spherical, elliptical, hemispherical, polygonal, etc. As whole body can be treated uniformly by making it into a sheet form, it is more preferable when treating the whole body.

In atmospheric pressure changing therapy, pressurizing the human body from the outside absorbs extra-cellular fluid in capillary vessels, increases blood circulation in the bone, internal organs, deep regions such as the retina, and improves cardiac function. In addition, it has been confirmed that as renal blood flow increases, excretion of surplus moisture in the body is promoted, that pressurization promotes water accumulation in cartilage, and that basal metabolism increases.

Atmospheric pressure changing therapy is a method that a person enters into oxygen capsules of 1.1 to 1.4 atm for 30 to 90 minutes. More preferable atmospheric pressure is 1.1 to 1.3 atm, particularly preferably 1.15 to 1.25 atm, and most preferably 1.23 atm. A more preferable time is 40 to 90 minutes, particularly preferably 50 to 70 minutes, most preferably 55 to 65 minutes. In the case of an oxygen capsule, the oxygen concentration can be 21 to 100% (% by volume), but it is preferably 21 to 50%, more preferably 21 to 42%, more preferably 21 to 36%, because there is a possibility of explosion. The lower limit may be equal to or higher than the oxygen concentration in the atmosphere (21% by volume), and the atmosphere itself may be pressurized in the oxygen capsule without separately adding oxygen.

A cartilage regeneration promoting effect can be obtained by ingesting 500 mL to 1 L of the cartilage regeneration promoting composition of the present invention before performing hormesis and atmospheric pressure changing therapy. The timing of ingesting the cartilage regeneration promoting composition of the present invention is preferably immediately before the start of hormesis and atmospheric pressure changing therapy, for example, 1 hour before the start, more preferably 30 minutes to 1 hour before the start, more preferably 10 minutes to 30 minutes before the start, particularly preferably 5 minutes to 10 minutes before start, most preferably 0 minutes to 5 minutes before the start.

As a method of using the system of hormesis, atmospheric pressure changing therapy and the bone regeneration promotion composition, hormesis and atmospheric pressure changing therapy of the present invention are carried out twice to seven times a week. While these are performed, it is preferable to ingest approximately 500 mL to 1 L of bone regeneration promoting composition every day, including day when hormesis and atmospheric pressure changing therapy is not received.

The cartilage regeneration promoting composition of the present invention is characterized by containing hydrogen nano bubbles. Furthermore, it is preferable to contain oxygen nanobubbles and/or nitrogen nanobubbles. Preferably, it is a composition comprising an aqueous solution containing nanobubbles having a particle size of 30 micrometers or less containing 0.45 to 0.55 ppm of hydrogen, 10 to 12.5 ppm of oxygen and 7 to 8 ppm of nitrogen.

A preferable concentration of the gas contained in the composition of the present invention is 0.1 to 3.0 ppm of hydrogen, 5 to 20 ppm of oxygen, and 3 to 20 ppm of nitrogen, more preferably 0.3 to 1.0 ppm of hydrogen, 7 to 15 ppm of oxygen, and 4 to 15 ppm of nitrogen, more preferably, 0.4 to 0.6 ppm of hydrogen, 9 to 13 ppm of oxygen, and 5 to 10 ppm of nitrogen, particularly preferably 0.45 to 0.55 ppm of hydrogen, 10 to 12.5 ppm of oxygen and 7 to 8 ppm of nitrogen. Since these concentrations have corresponding effects at respective concentrations, any concentration of these concentrations will have the effect of the present invention as long as it falls within this range. In addition, ppm means part per million, which represents the concentration of dissolved gas.

The gas contained in the composition of the present invention has a particle size of greater than 0, preferably less than 30 micrometers, more preferably less than 20 micrometers, even more preferably less than 10 micrometers, even more preferably less than 1 micrometer, particularly preferably 100 nanometers or less, most preferably 30 nanometers or less.

The composition for promoting cartilage regeneration containing the nanobubbles of hydrogen, oxygen and nitrogen of the present invention can be produced by the method of Patent Document 4, for example. Since the composition for promoting cartilage regeneration of the present invention is the same as the MCP described in Patent Document 4 and its use is different, in the present specification, the composition for accelerating cartilage regeneration of the present invention may be referred to as MCP.

As a form of the composition containing hydrogen nanobubbles, a liquid is most preferable, but it may be in the form of tablets, capsules or the like which generate hydrogen nano bubbles. In addition, hydrogen, oxygen and nitrogen may be absorbed directly by lungs by entering a subject into the capsule and putting some special composition gas into the capsule.

The bone regeneration-promoting composition of the present invention can be combined with one or more therapies selected from the group consisting of hormesis, atmospheric pressure changing therapy, and magnetic treatment. For example, humans who can not enter capsules due to claustrophobia or the like are difficult to undergo atmospheric pressure changing therapy, so they can be treated with other combinations of hormesis and bone regeneration composition. If a person is highly susceptible to hormesis, irradiation of hormesis may be changed to weaker irradiation, or the person may be treated by bone regeneration promoting composition ingestion and atmospheric pressure changing therapy excluding hormesis. If a person has a bad ear and is sensitive to change in atmospheric pressure, the person may be treated with bone regeneration promoting composition and hormesis without atmospheric pressure changing therapy. Also, the person may be treated by combining magnetic treatment with them.

As magnetic treatment, for example, it may be treated by applying an alternating magnetic field. As an apparatus for applying an alternating magnetic field, for example, Soken IV type electromagnetical therapy machine manufactured by Soken Medical Co., Ltd. is preferably used, the present invention is not limited thereto and any device capable of promoting bone regeneration by combining with any one of the bone regeneration composition, hormesis and atmospheric pressure changing therapy of the present invention, or with a plurality of combination of these may be used. The intensity of the applied magnetic field is preferably 100 to 1000 gauss, more preferably 300 to 800 gauss, and even more preferably 370 to 800 gauss. The time for applying magnetism is preferably 10 to 50 minutes, more preferably 10 to 40 minutes, further preferably 10 to 30 minutes. It is preferable to apply magnetism about 1 to 3 times per day. However, the present invention is not limited thereto, and the magnetic intensity, the time, and/or the number of times may be appropriately changed according to the age and symptoms of the patient. In brief, conditions can be set as appropriate within a range that bone regeneration and cartilage regeneration can be promoted, and that does not adversely affect the human body.

According to the present invention, fractures, necrosis, defects and the like of the bone head portion, the bone neck portion, the medial condyle, the joint, the cartilage and the like can be particularly suitably treated.

The cartilage regeneration promoting composition according to the present invention can be used for terrestrial or aquatic vertebrate animals Terrestrial vertebrates include, for example, humans, monkeys, cows, horses, pigs, sheep, goats, dogs, cats, rodents including ferrets, mice and rats, birds including chickens, geese and turkeys, the marsupials, reptiles and the like. As aquatic vertebrate animals, for example, fish and amphoterics can be listed and the like.

Examples of the cartilage regenerated by the cartilage regeneration promoting composition of the present invention include hyaline cartilage, fibrocartilage, elastic cartilage and the like. Examples of hyaline cartilage include articular cartilage, growth cartilage, tracheal cartilage, thyroid cartilage and the like. Examples of the fibrocartilage include an intervertebral disk, a pubic symphysis, a semilunar cartilage, an articular disk, and the like. Examples of the elastic cartilage include auricular cartilage and cartilage of the throat lid. The cartilage regenerated by the cartilage regeneration-promoting composition of the present invention is preferably hyaline cartilage, growth cartilage (promotion of leg extension of dwarfism) present in joints.

The composition for promoting cartilage regeneration according to the present invention can be used without limitation for various applications as long as the cartilage regeneration promoting effect is effectively available, it is particularly useful for treating diseases related to cartilage accompanied by dysfunction due to degeneration or destruction of cartilage, or hypoplasia and malformation of congenital cartilage.

Examples of such diseases include, but are not limited to, osteoarthritis, idiopathic femoral head necrosis, knee femoral cartilage defects, femoral internal condyle cartilage deficiency, patellofemoral arthritis, chronic rheumatoid arthritis, osteochondritis dissecans, damage of articular cartilage, articular cartilage calcification, intervertebral disc degeneration, anotia, microtia cartilage deficiency, cartilage defect in cartilage, achondroplasia (dwarfism), osteolysis surrounding the prosthetic device, temporomandibular disorder, or periodontal diseases accompanied by dissolution or destruction of alveolar bone, etc. In addition, as the composition for promoting cartilage regeneration according to the present invention can promote temporary bone formation in the repair process for trauma such as bone fracture or bone damage or resection due to surgery, for example, it is useful for restoration and regeneration promotion of intractable fractures and bone defects. The composition for promoting cartilage regeneration according to the present invention can also be used together with cartilage cells for transplantation in cartilage repair by cartilage cell transplantation into a cartilage defect site. Prior to the chondrocyte implantation, chondrocytes used for transplantation can also be grown in a culture medium to which the composition for promoting cartilage regeneration according to the present invention is added. Furthermore, the composition for promoting cartilage regeneration according to the present invention can also be used together with an implant for promoting implant engraftment when wearing implants in tissues or organs lost due to illness or injury.

The composition for promoting cartilage regeneration according to the present invention can be administered by various administration methods, for example, orally or parenterally. Preferably, it is oral administration, but when the application site is local, it can also be administered locally. By local administration, growth and increase of cartilage other than the administration site can be suppressed. As a composition for promoting cartilage regeneration for topical administration, it is preferable to administer a site where a cartilage tissue is present, for example, it is particularly preferable for intraarticular administration. Examples of the joint include a temporomandibular joint, a shoulder joint, an elbow joint, a hip joint, a knee joint, a fracture part (a part where a callus can be formed by a fracture), and the like.

The composition for promoting cartilage regeneration according to the present invention can be produced into various preparations together with a pharmacologically acceptable carrier. Examples of the form of the preparation include oral liquid preparations such as elixirs or syrups, for example, injections (for example, injections such as subcutaneous injections, intravenous injections, intramuscular injections, intraperitoneal injections, drip infusions), external preparations (for example, ointment, poultice, cream, paste, lotion, liniment, etc.) and the like. These preparations may be release-controlled preparations (e.g., sustained-release microcapsules) such as immediate-release preparations or sustained-release preparations. The formulation can be produced by a method commonly used in the field of formulation technology, for example, a method described in the Japanese Pharmacopoeia.

Idiopathic Osteonecrosis of Femoral Head (ION) is an illness of the hip joint, in which the bone tissue of the femoral head at the upper end of the femur necroses and in which the joints is deformed or destroyed, of which the cause is not clear. It is designated as a specific disease of the Ministry of Health, Labor and Welfare.

Osteoarthritis is a disease caused by joint deformity. It is understood that joints are sore from aging and excessive use of the knee. Typical joints to develop are knee joints, hip joints, ankle joints, etc. and occur frequently in joints subject to weight bearing. It also develop and cause pain in shoulder joints, elbow joints, wrist joints, finger joints, spinal facet joints. Knee femoral cartilage defect is a disease in which the cartilage of the knee femur is damaged due to an accident or the like and is defective. The medial femoral condyle bone defect is a disease in which the cartilage of the medial femoral condyle which is the inside part of the thigh bone among the thigh bones constituting the knee joint is defective.

HOMBD therapy consists of initials of hormesis, oxygen pressure changing therapy, mitochondrial power (trade name of composition for promoting bone regeneration), body therapy, diet. It is possible to regenerate cartilage just by drinking 500 ml per day of the bone regeneration promoting composition of the present invention, and by entering the space of hormesis and space pressure changing therapy twice a week, about one hour/once.

This breaks the common sense of modern medicine that damaged cartilage is not regenerated, thereby it has a possibility as a completely new treatment for aging pathology raising the patient's quality of life (QOL).

Hereinafter, the present invention will be described by examples, but the present invention is not limited by any of these examples.

### EXAMPLES

(Hormesis+Space Pressure Changing Therapy+Nanobubble Water Combination Therapy)

A patient who ingested 500 mL of the cartilage regeneration-promoting composition (MCP) of the present invention was placed on a hormesis sheet (20 to 28μ sievert/hr) laid in an oxygen capsule and placed in an oxygen capsule (Phoenix I 2 A 7, Kobe Medicare Co., Ltd.) with 1.23 atm for 1 hour. The treatment was done twice a week. The effect was measured by photographing the affected part by MRI (nuclear magnetic resonance imaging) and by processing it with software. Since moisture content is shown colored in MRI mapping images, as the cartilage decreases, the area indicated by green and blue decreases and red areas increases. Conversely, when cartilage is regenerated, the areas of the green and blue parts increase on the image. Oxygen can be produced with an oxygen concentrator and in an oxygen capsule it can be treated, for example, with an oxygen concentration of 36 to 42%.

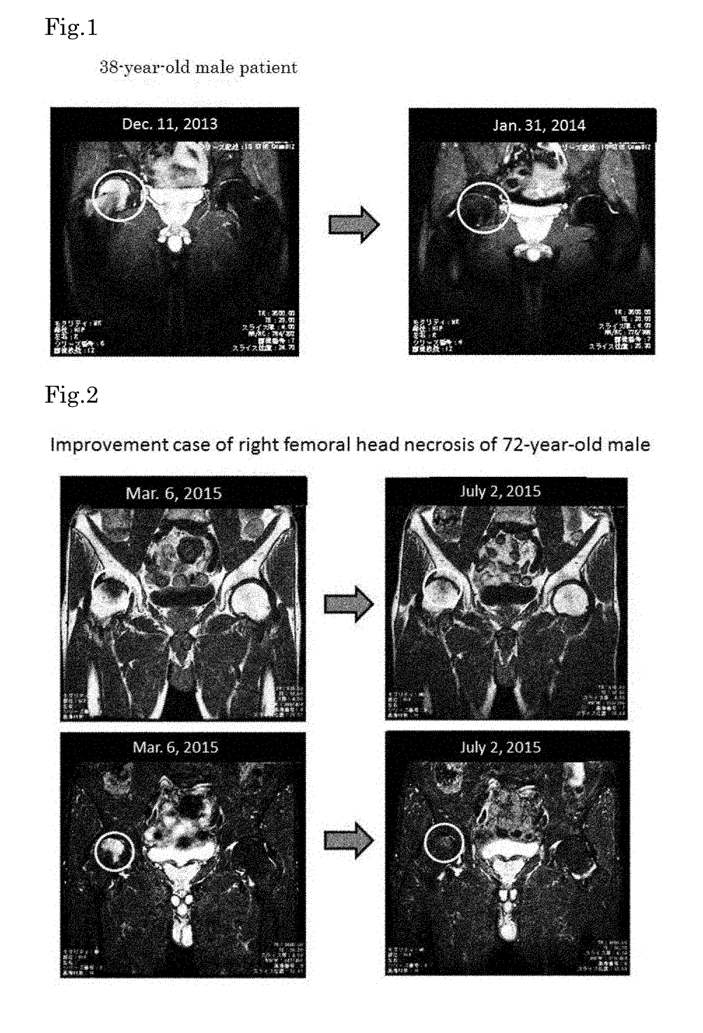

When a 38-year-old male patient drove off a slope by driving a passenger car, a signal-ignored car entered from the right side at the intersection. He suddenly applied a braking, but did not make it in time. It collided and he got injured. After this accident, pain in the right hip joint continued, and no fracture was recognized on X-ray, but it was diagnosed as headache necrosis due to fracture under the femoral head by MRI-CT examination. He was recommended for surgery, but he wanted conservative treatment. Therefore, the composition for promoting bone regeneration of the present invention was used in combination with hormesis therapy and atmospheric pressure changing therapy. The location where the head necrosis had occurred (the white portion inside the white circle in FIG. 1) was on day 51(photograph of right side) returned to nearly the original state, and organization restoration in a short time was observed (FIG. 1).

72-Year-Old Man⋅Improvement Case of Right Femoral Head Necrosis

On March 6, the femoral head had been necrotized, but treatment with the composition for promoting bone regeneration in combination with hormesis and atmospheric pressure changing therapy of the present invention resulted in disappearance of nearly all necrotic part on July 2 after about 4 months (FIG. 2). The figure below is a black and white inversion. The white part in the white circle of the lower left picture becomes black in the major part of the lower right on July 2.

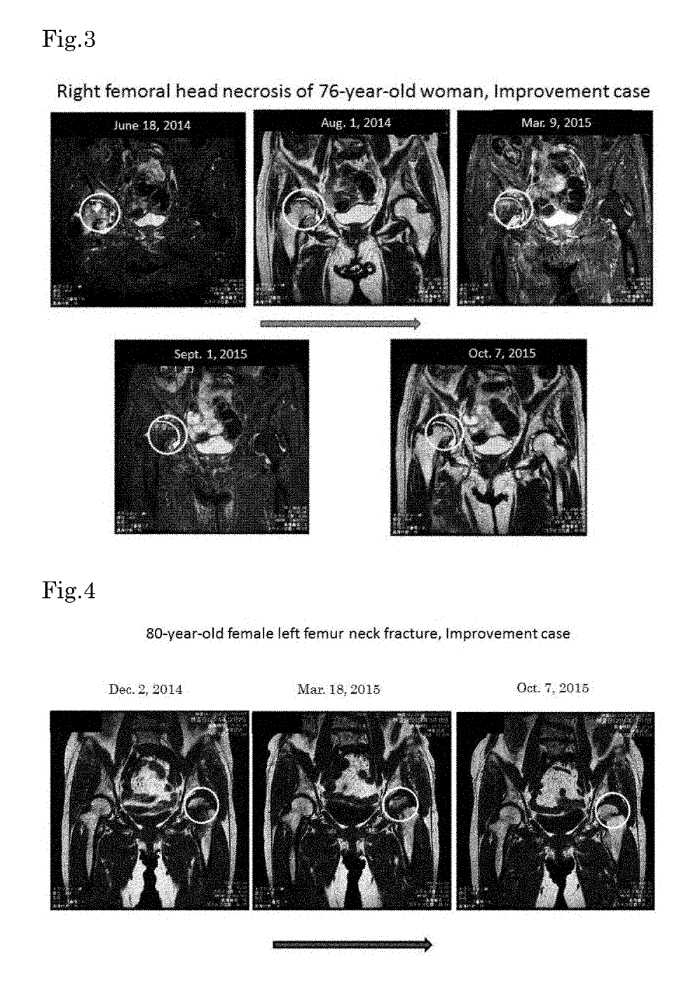

FIG. 3 shows a improvement case of a 76-year-old woman with necrotizing and ameliorating head of right femoral head. In the upper left picture, a semicircular thin part (cartilage) surrounding the head is ruptured but it recovers as time goes by, and connected in the photograph on the lower right almost completely like a semicircular string.

80-Year-Old Female Left Femur Neck Fracture/Improvement Example

On 2 Dec. 2014, the bone fractured and the bones were far apart, but after treated with the combination of the bone regeneration promoting composition of the present invention in combination with hormesis and atmospheric pressure changing therapy, In the white circle on Dec. 2, 2014 there are many black parts under the bone neck, improvement was seen on Mar. 18, 2015, on October 7 the same year the bone neck inside the white circle It turns out that the whole area becomes whitish and it is repaired. (FIG. 4)

27-Year-Old Male Left Femoral Head Necrosis/Improvement Case

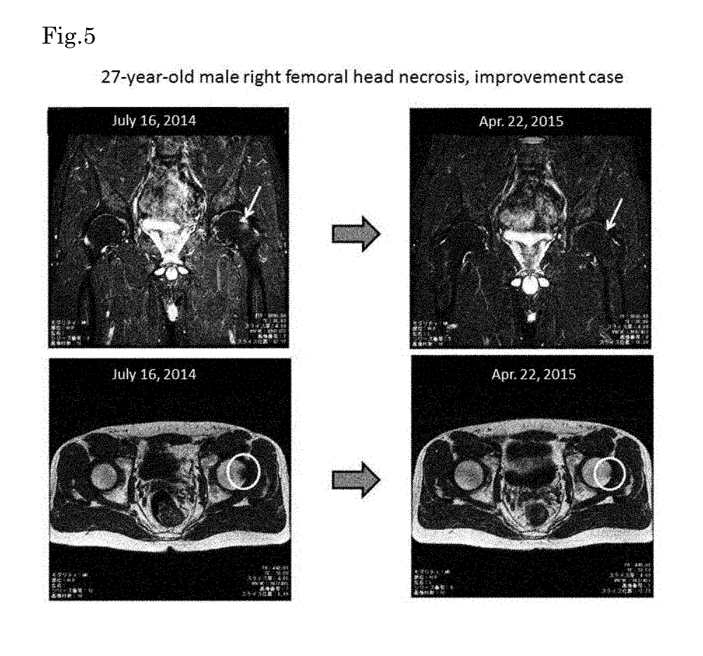

On Jul. 16, 2014, the upper right part of the femoral head is necrosed and appears white (the arrow part of the photo in the upper picture), but after treated in combination with the bone regeneration promoting composition of the present invention and hormesis, atmospheric pressure changing therapy, on Apr. 22, 2015 the white part is almost gone (picture in the upper part of FIG. 5). The image on the top removed fat and the bottom image was taken to make fat visible. In the lower photograph in FIG. 5, also, the white part in the white circle on the right side is more white than the left, which shows that the necrosis of the femoral head has been improved (black and white is reversed from the above picture).

82-Year-Old Female Right Knee Osteoarthritis/Improvement Case

Articular Cartilage Photography by MRI (CT2-MAP)

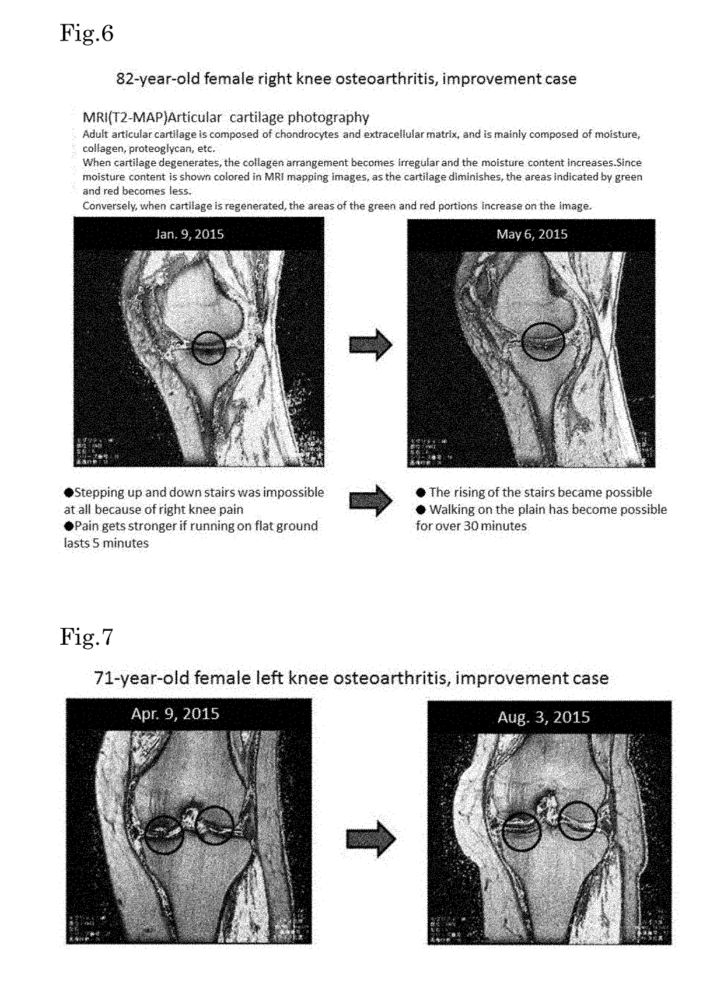

The moisture content of the cartilage part was small in January, September 2015, but after treated with the combination of the bone regeneration promoting composition of the present invention in combination with hormesis and atmospheric pressure changing therapy, On May 6 of the same year, the moisture content increased, the parts between the left and right were connected, and it was found that the cartilage was regenerated (FIG. 6). As a result, going up and down the stairs was impossible at all because of right knee pain, but becomes possible to go up and down the stairs. Although the pain had become stronger after 5 minute walk on the flat areas, walking for more than 30 minutes became possible.

Improvement Case of 71-Year-Old Female Left Knee Osteoarthritis

On Apr. 9, 2015, moisture was small in the part inside the circle. After treated with the bone regeneration promoting composition of the present invention combined with hormesis and atmospheric pressure changing therapy, on August 3 the same year, moisture content has recovered (FIG. 7).

Improvement Case of 75-Year-Old Female Osteoarthritis Medial Articular Cartilage Defect

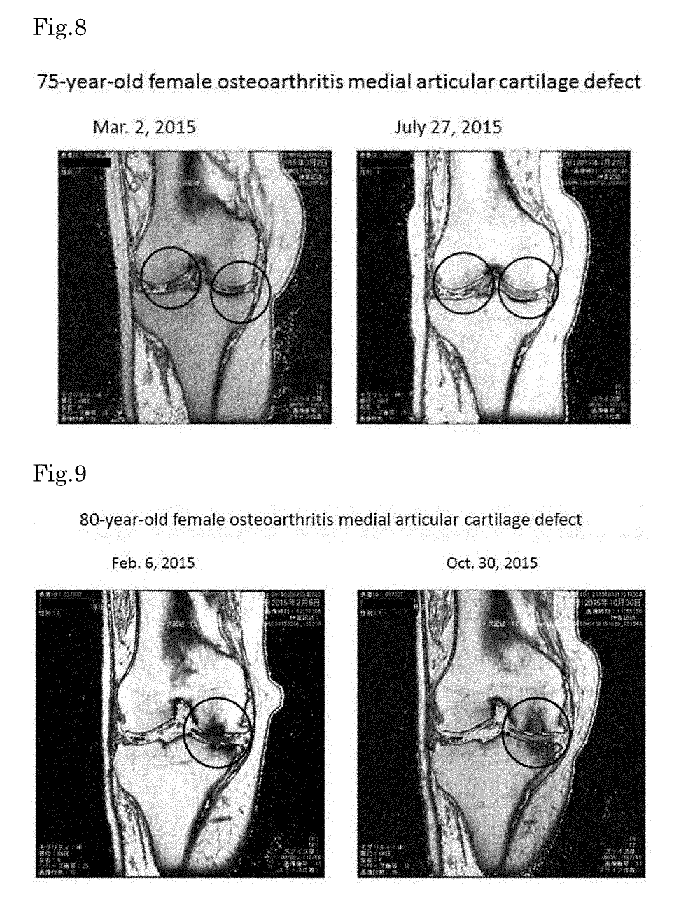

On Mar. 2, 2015, the moisture content in the circle was small, but treatment with the composition for promoting bone regeneration of the present invention in combination with hormesis and atmospheric pressure changing therapy resulted in recovery of moisture content on July 27 of the same year (See a circle on the right side of FIG. 8).

Improvement Cases of 80-Year-Old Female Osteoarthritis Medial Articular Cartilage Defect

On Feb. 6, 2015, the moisture content in the circle was small, but treatment with the bone regeneration promoting composition of the present invention in combination with hormesis and atmospheric pressure changing therapy resulted in bone regeneration on October 30 of the same year, and the moisture content has also recovered (FIG. 9).

Improvement Case of 49-Year-Old Male Right Knee Femoral Cartilage Defect

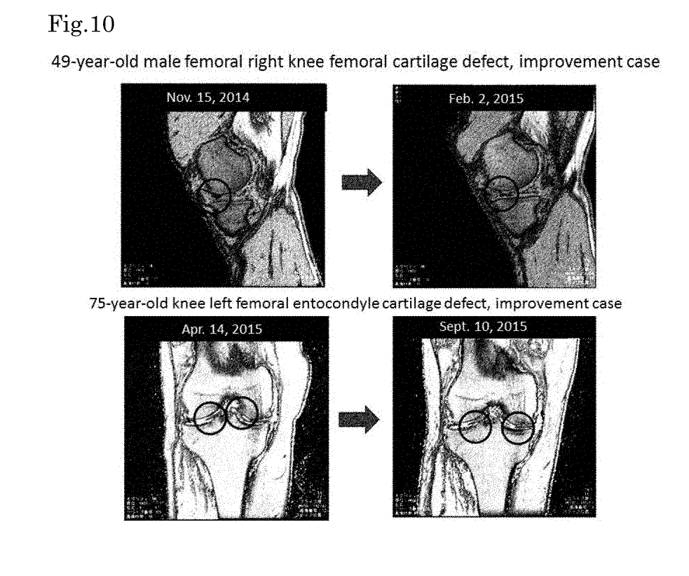

On Nov. 15, 2014, the moisture content in the circle was small, but treatment with the bone regeneration promoting composition of the present invention in combination with hormesis and atmospheric pressure changing therapy resulted in a moisture content recovery on Feb. 2, 2015 (FIG. 10, top).

Improvement Case of 75-Year-Old Knee Left Femoral Cartilage Defect

On Apr. 14, 2015, the moisture in the circle had been decreasing. However, after treated with the composition for promoting bone regeneration of the present invention combined with hormesis and atmospheric pressure changing therapy, on September 10, the same year, moisture content recovered (FIG. 10, bottom).

Improvement Case of 84-Year-Old Female Left Knee Osteoarthritis

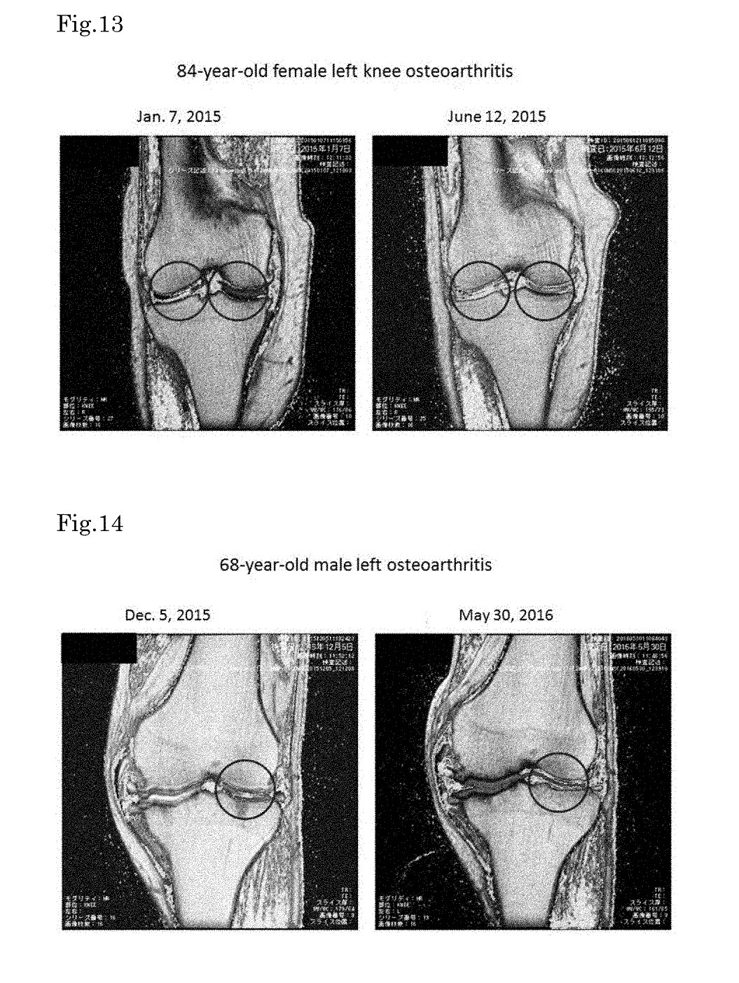

On Jan. 7, 2015 the moisture content in the circle had been decreasing, but after treated with the combination of the bone regeneration promoting composition of the present invention in combination with hormesis and atmospheric pressure changing therapy, on June 12, the same year the moisture content was recovered (See the circle on the right of FIG. 13).

Improvement Case of 68-Year-Old Male Left Knee Osteoarthritis

On Dec. 5, 2015, moisture in the circle had been decreasing, but after treated with the composition for promoting bone regeneration of the present invention in combination with hormesis and atmospheric pressure changing therapy, on May 30, 2016, moisture content was recovered (see the circle on the right of FIG. 14).

Improved Cases of 75-Year-Old Female Patellofemoral Arthritis

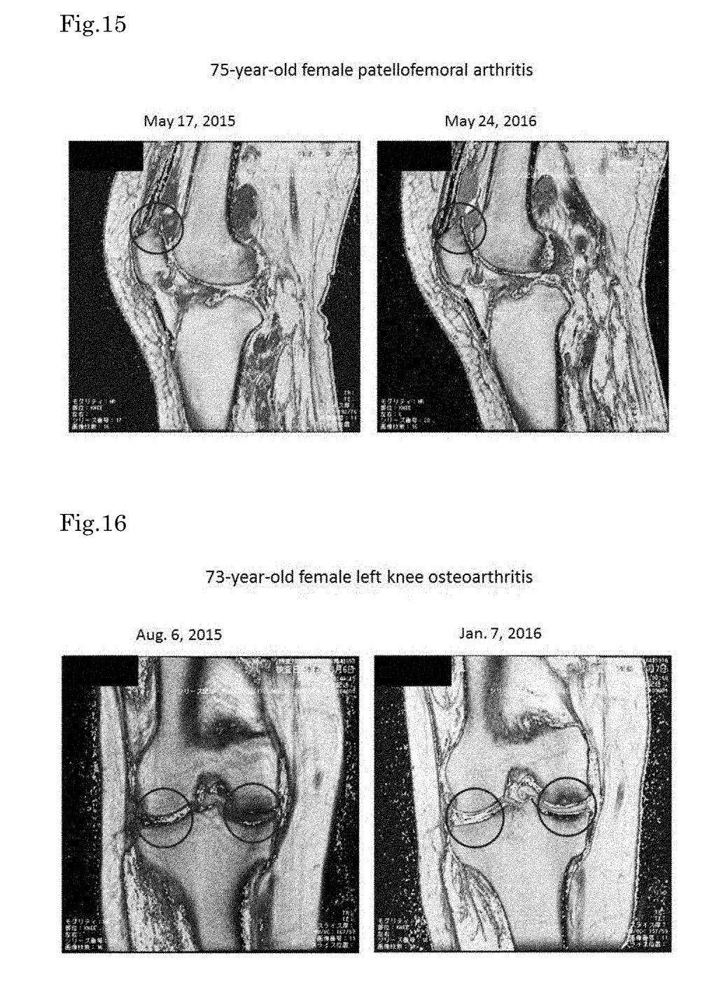

On May 17, 2015, the moisture in the circle had been decreasing, but after treated with the combination of the bone regeneration promoting composition and hormesis, atmospheric pressure changing therapy of the present invention, on May 24, 2016 moisture content recovered (see the circle on the right in FIG. 15).

Improvement Case of 73-Year-Old Female Left Knee Osteoarthritis

On Aug. 6, 2015, moisture in the circle had been decreasing, but after treated with a combination of the bone regeneration promoting composition of the present invention, hormesis, and atmospheric pressure changing therapy, on Jan. 7, 2016, moisture content recovered (see the circle on the right of FIG. 16).

Improved Case of 83-Year-Old Female Left-Knee Osteoarthritis

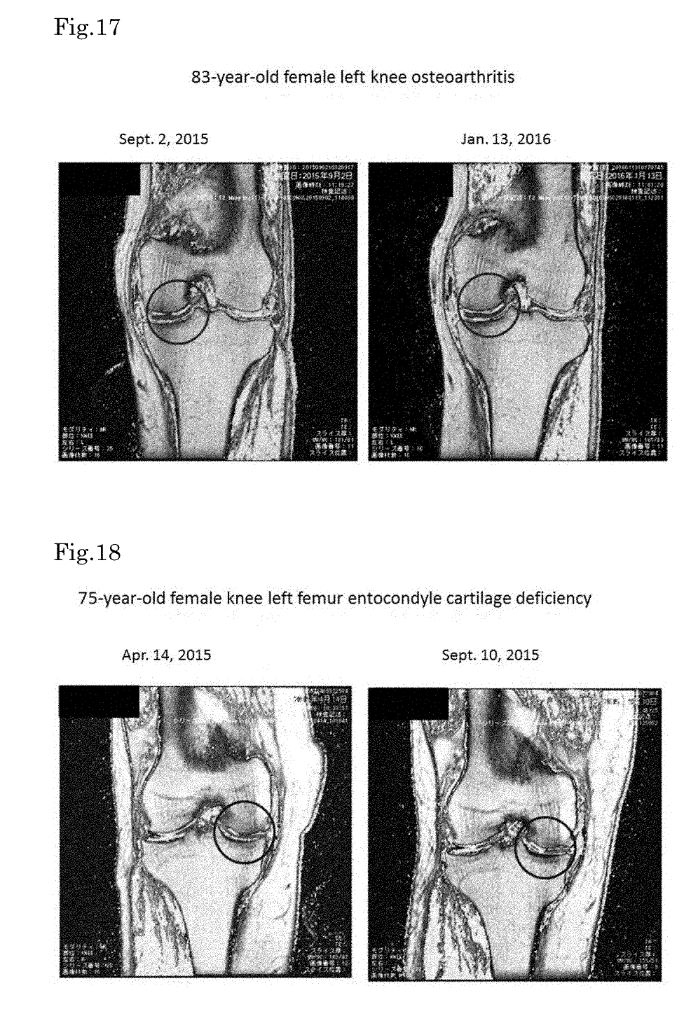

On Sep. 2, 2015, the moisture in the circle had decreased, but after treated with the composition for promoting bone regeneration of the present invention in combination with hormesis and atmospheric pressure changing therapy to treat, on Jan. 13, 2016 moisture content recovered (see the circle on the right of FIG. 17).

Improved Case of 75-Year-Old Female Left Femur Entocondyle Deficiency

On Apr. 14, 2015, the moisture in the circle had been decreasing. However, after the composition for promoting bone regeneration of the present invention was combined with hormesis and atmospheric pressure changing therapy to treat, on September 10, the same year, moisture content was recovered (See the circle on the right of FIG. 18).

Improvement Case of 87-Year-Old Female Right Femoral Head Necrosis

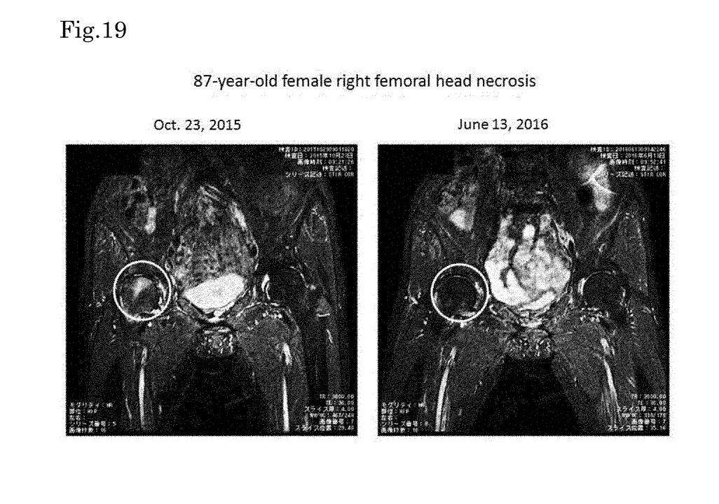

On Oct. 23, 2015, the moisture in the circle had decreased. But after treatment with the composition for promoting bone regeneration in combination with hormesis and atmospheric pressure changing therapy of the present invention, the moisture content recovered on Jun. 13, 2016 (see the circle on the right of FIG. 19).

From the above, it was revealed that according to the present invention, the moisture content of the injured site increased and cartilage regeneration occurred.

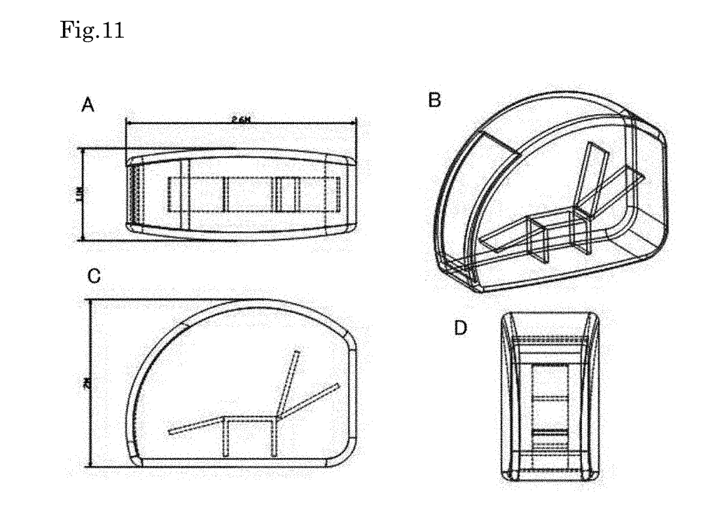

FIG. 11 is a schematic diagram of an apparatus for carrying out hormesis, atmospheric pressure changing therapy. Because there are some patients who can not enter the horizontal capsule, they are made to be able to receive treatment sit in a chair so that they can receive. It is also possible to recline if necessary to receive treatment in a horizontal state.

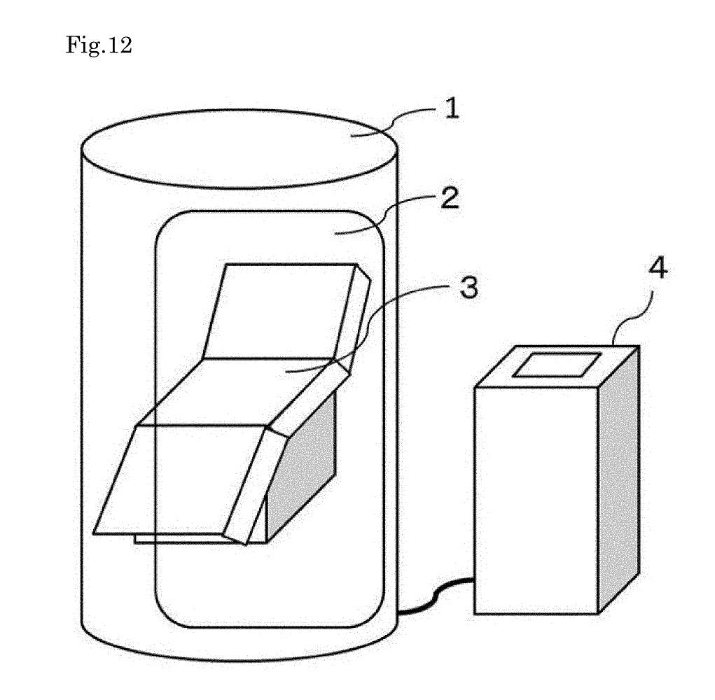

FIG. 12 shows another form of apparatus that performs hormesis, atmospheric pressure changing therapy. In this case, it is a device which increases the therapeutic effect by feeding a gas of a special composition into the capsule in addition to hormesis, atmospheric pressure changing therapy, ingestion of bone regeneration promoting agent of the present invention. The gas generator 4 produces a mixed gas of hydrogen, oxygen and nitrogen and sent them to the capsule, so that the treatment effect is further enhanced by absorbing the mixed gas also from the lungs. Also, this capsule is equipped with a device to generating magnetic and can treat with magnetism (and/or hormesis and space pressure therapy).

### INDUSTRIAL APPLICABILITY

The present invention can be utilized in the medical and health industries.

### DESCRIPTION OF THE REFERENCE NUMERALS

1. capsule
2. Sliding door
3. Reclining chair
4. Gas generator

1. A method for promoting bone regeneration comprising a step of ingesting a composition for bone regeneration promotion comprising nanobubbles of hydrogen, and/or oxygen and nitrogen.

2. The method for promoting bone regeneration according to claim 1, further comprising one or more step(s) selected from a step for hormesis and a step for atmospheric pressure changing therapy.

3.-5. (canceled)

6. A cartilage regeneration system comprising a composition for bone regeneration promotion comprising nanobubbles of hydrogen, and/or oxygen and nitrogen, an apparatus for hormesis and a device for atmospheric pressure changing therapy.

7. The cartilage regeneration system according to claim 6, wherein the apparatus for hormesis is a hormesis sheet.

8. The method for promoting bone regeneration according to claim 2, comprising a method for operation of the atmospheric pressure changing therapy, which pressurize to 1.1-1.5 atm, wherein a ratio of hydrogen:oxygen:nitrogen is 0.1 to 4.0:20 to 50:46 to 79.1.

9. The cartilage regeneration system according to claim 6, wherein the said device for atmospheric pressure changing therapy comprising a sealable capsule device, comprising
a mean for supplying mixed gas of hydrogen, oxygen and nitrogen, and
a mean regulating an atmospheric pressure in the capsule.
